(19) United States
(12) Patent Application Publication
(10) Pub. No.: US 2022/0392608 A1
(43) Pub. Date: Dec. 8, 2022

(54) PATIENT AUTHENTICATION AND REMOTE MONITORING FOR PULSED ELECTROMAGNETIC FIELD SYSTEMS

(71) Applicant: Regenesis Biomedical, Inc., Scottsdale, AZ (US)

(72) Inventors: Manuel J. FERRER HERRERA, Scottsdale, AZ (US); Donald B. TATE, Scottsdale, AZ (US); Ryan BINETTE, Scottsdale, AZ (US); Thomas I. EISIMINGER, JR., Scottsdale, AZ (US); Scott A. ROBEY, Scottsdale, AZ (US)

(21) Appl. No.: 17/832,444

(22) Filed: Jun. 3, 2022

Related U.S. Application Data
(60) Provisional application No. 63/196,672, filed on Jun. 3, 2021.

(51) Int. Cl. G16H 20/40; A61B 5/117; G16H 10/60; A61N 2/00

(52) U.S. Cl. CPC G16H 20/40; A61B 5/117; G16H 10/60; A61N 2/004

(57) ABSTRACT

A method and apparatus for pulsed electromagnetic field therapy to one or more patients is disclosed. In some embodiments, a pulsed electromagnetic field (PEMF) therapy device may include a biometric authentication device to verify the identity of the patient. Prior to receiving treatment, the identity of the patient may be verified though the biometric authentication device. After the patient's identity is verified, the PEMF therapy device may receive a PEMF treatment plan for the patient that includes a PEMF applicator type, a treatment duration, and an energy level associated with the PEMF treatment.

### CLAIM OF PRIORITY

This application claims priority to U.S. provisional patent application No. 63/196,672, titled “PATIENT AUTHENTICATION AND REMOTE MONITORING FOR PULSED ELECTROMAGNETIC FIELD SYSTEMS,” filed on Jun. 3, 2021, and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Pulsed electromagnetic fields (PEMF) have been described for treating therapeutically resistant problems of both the musculoskeletal system as well as soft tissues. PEMF typically includes the use of time-varying magnetic fields. For example, PEMF therapy has been used to treat non-union bone fractures and delayed union bone fractures. PEMF therapy has also been used for treatment of corresponding types of body soft tissue injuries including chronic refractory tendinitis, decubitus ulcers (e.g., diabetic ulcers), wounds, and ligament, tendon injuries, osteoporosis, and Charcot foot. During PEMF therapy, an electromagnetic transducer coil is generally placed in the vicinity of the injury (sometimes referred to as the “target area”) such that pulsing the transducer coil will produce an applied or driving field that penetrates to the underlying tissue.

Treatment devices emitting magnetic and/or electromagnetic energy offer significant advantages over other types of electrical stimulators because magnetic and electromagnetic energy can be applied externally through clothing and wound dressings, thereby rendering such treatments completely non-invasive. Moreover, published reports of double-blind placebo-controlled clinical trials utilizing a RF transmission device suggest that this ancillary treatment device significantly reduces wound healing time for chronic pressure ulcers as well as for surgical wounds. Studies using Dermagen, a magnetic device manufactured in Europe which produces a low frequency magnetic field, have demonstrated significant augmentation of healing of venous stasis ulcers. Additionally, it has been shown for groups of patients treated with electromagnetic energy, that 50% fewer patients of that treatment group develop reoccurring pressure ulcers, compared to control patients, suggesting that electromagnetic energy treatments impart some resistance to the reoccurrence of chronic wounds, such as pressure ulcers. Electromagnetic energy may also be useful as a preventative strategy. Analysis of the effects of electromagnetic energy on the treatment of pressure ulcers show that this treatment, by reducing healing time by an average of 50%, results in significant reductions in the costs associated with wound management.

A patient may receive PEMF treatment from PEMF therapy devices. Typically, each patient may have his or her own unique prescribed therapy. Ensuring that the patient's prescribed therapy is delivered correctly to the patient may be difficult, particularly if the PEMF treatment device is shared with other patients. Furthermore, it may be difficult to limit access to the PEMF treatment device to authorized users.

### SUMMARY OF THE DISCLOSURE

This disclosure relates generally to pulsed electromagnetic field (PEMF) systems, apparatuses, and methods. In particular, the disclosure relates to higher-powered pulsed electromagnetic field (PEMF) applicator systems.

In general, described herein are pulsed electromagnetic field (PEMF) apparatuses (e.g., devices and systems) including a PEMF therapy device configured to generate a pulsed current signal and a biometric authentication device to verify a patient's identity. In particular, apparatuses are described herein that may be configured determine whether a verified patient is attempting to use the PEMF therapy device. If the patient's identity is verified, then the PEMF therapy device may deliver PEMF therapy through attached or coupled PEMF applicators.

In some examples, the PEMF therapy device may communicate with a treatment coordination server to receive the patient's PEMF treatment plan. In this manner, the correct PEMF treatment is delivered to the correct patient.

One aspect of subject matter described herein may be implemented in a method for controlling operations of a high-power pulsed electromagnetic field (PEMF) therapy device. The method may include verifying a patient's identity via a biometric subsystem of the PEMF device, transmitting the verified patient's identity to a treatment coordination server, receiving a PEMF treatment plan associated with the transmitted patient's identity from the treatment coordination server, and delivering a PEMF treatment to the patient.

In some examples, the biometric subsystem may be a fingerprint scanner, a retina scanner, a facial recognition device, a voice recognition device, a hand scanner, or a combination thereof. In some other examples, the transmitting the verified patient identity may include encrypting the verified patient identity. Further, receiving the PEMF treatment plan may include decrypting the PEMF treatment plan from the treatment coordination server.

In some examples, the method may include verifying one or more PEMF therapy applicators coupled to the PEMF device prior to delivering the PEMF treatment. Furthermore, the PEMF treatment may not delivered when the one or more PEMF therapy applicators coupled to the PEMF therapy device are not included in the PEMF treatment plan.

In some examples, the method may include collecting patient data via the PEMF therapy device. Further, the patient data may include self-assessment data, pain data, patient pulse rate, patient temperature, patient sleep cycle information, or a combination thereof. Additionally, the method may include transmitting the patient data to the treatment coordination server. The method may also include updating the PEMF treatment plan based on received patient data.

In some examples, the method may include disabling the PEMF device based on verified patient's identity.

Another innovative aspect of the subject matter described in this disclosure may be implemented in a system. The system may include a treatment coordination server configured to store a patient's pulsed electromagnetic field (PEMF) treatment plan. The system may also include a PEMF therapy device configured to verify a patient's identity via a biometric subsystem of the PEMF device, transmit the verified patient's identity to the treatment coordination server, receive a PEMF treatment plan associated with the patient's identity from the treatment coordination server, and deliver a PEMF treatment to the patient.

In some examples, the biometric subsystem may be a fingerprint scanner, a retina scanner, a facial recognition device, a voice recognition device, a hand scanner, or a combination thereof. In some other examples, the PEMF therapy device may be configured to encrypt the verified patient identity. In still other examples, the PEMF treatment plan may include a PEMF applicator type, a therapy duration, and an energy level associated with the PEMF therapy.

In some examples, the PEMF therapy device may be further configured to decrypt the PEMF treatment plan from the treatment coordination server. In some other examples, the PEMF therapy device may be further configured to verify one or more PEMF therapy applicators coupled to the PEMF therapy device prior to delivery of the PEMF treatment. Further, the PEMF therapy device may be further configured to not deliver the PEMF treatment when the one or more PEMF therapy applicators coupled to the PEMF therapy device are not included in the PEMF treatment plan.

In some examples, the PEMF therapy device may be configured to collect patient data. Furthermore, the patient data may include self-assessment data, pain data, patient pulse rate, patient temperature, patient sleep cycle information, or a combination thereof. Still further, the PEMF therapy device may be configured to transmit the patient data to the treatment coordination server. The treatment coordination server may be configured to update the PEMF treatment plan based on received patient data.

In some examples, the PEMF therapy device may be configured to disable PEMF therapy based on the verified patient's identity.

Another innovative aspect of the subject matter described in this disclosure may be implemented in a non-transitory computer-readable storage medium. The storage medium may store instructions that, when executed by one or more processors of a pulsed electromagnetic field (PEMF) therapy device, cause the PEMF therapy device to verify a patient's identity via a biometric subsystem of the PEMF device, transmit the verified patient's identity to a treatment coordination server, receive a PEMF treatment plan associated with the transmitted patient's identity from the treatment coordination server, and deliver a PEMF treatment to the patient.

In some examples, the biometric subsystem may be a fingerprint scanner, a retina scanner, a facial recognition device, a voice recognition device, a hand scanner, or a combination thereof. In some other examples, execution of the instructions to transmit the verified patient's identity may further include instructions to encrypt the verified patient identity. In still other examples, the PEMF treatment plan may include a PEMF applicator type, a therapy duration, and an energy level associated with the PEMF therapy.

In some examples, execution of the instructions may cause the PEMF therapy device to decrypt the PEMF treatment plan from the treatment coordination server. In some other examples, execution of the instructions may cause the PEMF therapy device to verify one or more PEMF therapy applicators coupled to the PEMF therapy device prior to delivery of the PEMF treatment. Furthermore, execution of the instructions may cause the PEMF therapy device to not deliver the PEMF treatment when the one or more PEMF therapy applicators coupled to the PEMF therapy device are not included in the PEMF treatment plan.

In some examples, execution of the instructions may cause the PEMF therapy device to collect patient data. Furthermore, the patient data may include self-assessment data, pain data, patient pulse rate, patient temperature, patient sleep cycle information, or a combination thereof. Still further, execution of the instructions may cause the PEMF therapy device to transmit the patient data to the treatment coordination server. Additionally, the PEMF treatment plan may be updated based on received patient data.

In some examples, execution of the instructions may cause the PEMF therapy device to disable PEMF therapy based on the verified patient's identity.

In general, these methods and apparatuses may be configured to identify the identity of the patient to use the PEMF device (using, e.g., a biometric input from the patient and/or other identity-confirming information such as passcodes, etc.) as well as the identity and validity of the one or more applicators associated with the PEMF device. Thus, the PEMF device may transmit information (which may be encoded) to a remote treatment coordination server, including an indicator of the patient's identity (such as a biometric identifier), an indicator of the identity of the PEMF device that the patient is to use, and an authentication indicator for the one or more applicators to be used by the patient with the device. This information may be wireless transmitted to the treatment coordination system. The treatment coordination system may then confirm this three-way authentication (the user, the device and the applicators) and may provide an appropriate treatment plan that is specific to the user, the device and/or the applicators.

For example, described herein are methods of controlling operation of a pulsed electromagnetic field (PEMF) therapy device that include: receiving an indicator of a patient's identity via a biometric subsystem of the PEMF device; receiving an encrypted accessory authentication indicator for one or more applicators coupled to the PEMF device; transmitting the indicator of the patient's identity, an indicator of the PEMF therapy device identity, and the encrypted accessory authentication indicator to a treatment coordination server; receiving a PEMF treatment plan associated with the patient's identity from the treatment coordination server upon verification by the treatment coordination server that the patient identity is linked to the one or more applicators and to the PEMF device; and delivering the PEMF treatment to the patient.

In any of the methods described herein, the patient's identity may be verified in the PEMF device using the indicator of the patient's identity before transmitting the indicator of the patient's identity to the treatment coordination server. Alternatively (or in some case, additionally), the patient's identity may be verified in the treatment coordination server using the indicator of the patient's identity.

Any of the information transmitted between the PEMF device and the treatment coordination system may be encoded. For example, transmitting the indicator of the patient's identity may include encrypting the indicator of the patient's identity.

In any of these methods, the received PEMF treatment plan may be specific to the PEMF applicator and may include a therapy duration, and/or an energy level associated with the PEMF therapy. The PEMF treatment plan may further comprise decrypting the PEMF treatment plan.

Any of these methods may include receiving a notification that the patient's identity is not linked to the one or more applicators and/or to the PEMF device. The PEMF device may be configured to display a message indicating that there is a failure to verify the identity of the patient and/or the applicators with the PEMF device. The PEMF device may also or additionally lock or prevent the application of a treatment plan until unlocked. In some cases, the PEMF device may indicate that the applicator(s) is/are incorrect, have expired, and/or need to be attached and/or replaced.

In general, any of these methods may be performed by a system as described herein, including a non-transitory computer-readable storage medium storing instructions that, when executed by one or more processors of a pulsed electromagnetic field (PEMF) therapy device, cause the PEMF therapy device to perform any of these methods.

The methods and apparatuses described herein may generally allow regulating and securing access to use prescribed medical devices. These methods and apparatuses may ensure a medical device that requires prescription use is only accessible to the users/patients the device has been prescribed for or authorized to use. In particular, the methods and apparatuses described herein are easier to use and may provide authentication that offers simplicity and remote support abilities to provide convenience to the users/patients of the PEMF device.

In addition, the methods and apparatuses described herein are particularly helpful, because they allow the apparatus (e.g., device and systems) to track users/patients activity and treatment progress, which may be stored, transmitted and/or used to modify the treatment program specific to that user/patient which can improve their outcomes further. For example, these apparatuses may collect electronic device data in combination with user/patient-provided data and provide users/patients with customized therapeutic treatments and/or recommended lifestyle changes that can improve or enhance the outcomes of their therapeutic treatment.

The methods and apparatuses (e.g., devices, systems, including PEMF devices) described herein may leverages biometric sensing, wireless communication, cybersecurity, and cloud computing technology to provide a higher level of value to the user/patient. Thus, described herein are multiple methods to protect the user/patient (these methods can be implemented/used on other medical devices and healthcare technologies as well) while they engage in a pulsed electro-magnetic field (PEMF) therapy treatment protocol. Although the methods and apparatuses described herein specifically include PEMF devices, other medical devices may be similarly used, including medical diagnostic and imaging devices, other therapeutic electrical and/or mechanical devices, etc. The methods and apparatuses described herein may also provide multiple ways to allow a clinician/caregiver to securely monitor and oversee the user/patient progress through the PEMF therapy protocol remotely/virtually via wireless communication and cloud computing.

Any of these methods and apparatuses may link the medical device (i.e., therapeutic PEMF device) to a specific patient who is authorized and has a prescription to utilize it exclusively. These methods and apparatuses may authenticate, including by biometric authentication, the patient authorized to use or access the medical device (i.e., therapeutic PEMF device) prior to each therapeutic treatment; as mentioned, this authorization may also include authorization of the applicator (e.g., PEMF applicator). The information (data) may be encrypted. For example, authentication of the applicator(s) may include decrypting encrypted information identifying the applicator(s). The applicator(s), e.g., therapy application pads, that are connected to the medical device (i.e., therapeutic PEMF device) may thereby be confirmed as genuine and used as intended per the prescription and the therapeutic treatment. The particular patient treatment (therapy) may also be customized based on the attached applicator, which may have different sizes and characteristics.

These methods and apparatuses may also authenticate and encrypt data as a secure method to obtain and aggregate (store) and/or transmit outcomes data and information related to the medical device (i.e., therapeutic device data) and the patient (e.g., physiological data), and may use the data and information to recommend lifestyle changes that can improve or enhance the outcomes of the therapeutic treatment.

In general, these methods and apparatuses may include wireless connectivity capability that may be used to perform remote software/firmware updates to the medical device (i.e., therapeutic device).

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a diagram of a PEMF system, according to some examples.

FIG. 2 depicts a PEMF therapy device.

FIG. 3 is a flowchart depicting one example method for providing a pulsed electromagnetic field therapy to a patient.

FIG. 4 is a flowchart depicting another example method for providing a pulsed electromagnetic field therapy to a patient.

FIG. 5 shows a block diagram of a PEMF therapy device that may be one example of the PEMF therapy device of FIG. 1 or the PEMF therapy device of FIG. 2.

### DETAILED DESCRIPTION

A pulsed electromagnetic field (PEMF) therapy device may include a biometric authentication device that may be used to limit the use of the PEMF therapy device to authorized users. In some examples, after a patient's identity is verified with the biometric authentication device, the PEMF therapy device may contact a treatment coordination server to receive details regarding the patient's PEMF therapy. In some other examples, a clinician may update the patient's PEMF therapy, and the updated therapy may automatically downloaded to the PEMF therapy device.

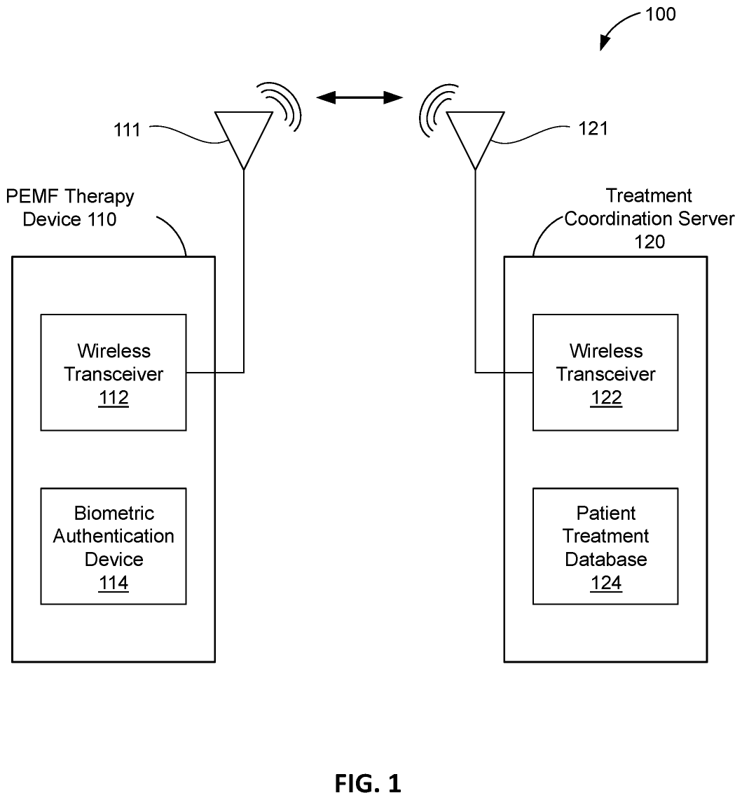

FIG. 1 is a diagram of a PEMF system 100, according to some examples. The PEMF system 100 may include a PEMF therapy device 110 and a treatment coordination server 120. The PEMF therapy device 110 may include an antenna 111, a wireless transceiver 112 and a biometric authentication device 114. The treatment coordination server 120 may include an antenna 121, a wireless transceiver 122 and a patient treatment database 124.

The PEMF therapy device 110 may generate and provide one or more magnetic and/or electromagnetic fields to a patient through one or more PEMF applicators (not shown). The magnetic and/or electromagnetic fields may provide a therapeutic effect for the patient in a non-invasive manner. For example, the PEMF therapy device 110 may be configured to provide high-power, pulsed electromagnetic field signals to one or more coupled applicators. The one or more applicators may be configured to deliver magnetic fields to a patient through one or more PEMF applicators that include one or more electromagnetic transducers.

The wireless transceiver 112 of the PEMF therapy device 110 may be coupled to the antenna 111. The wireless transceiver 112 may communicate with other devices through one or more wireless protocols. For example, the wireless transceiver 112 may be configured to communicate via wireless networks conforming to any of the IEEE 802.11 family of standards, cellular networks conforming to any of the LTE standards promulgated by the 3^{rd} Generation Partnership Project (3GPP) working group (e.g., 3G, 4G LTE, 5G LTE, or the like), WiMAX networks, Bluetooth networks, or the like. Additionally, or alternatively, the wireless transceiver 112 may be configured to communicate through any feasible wired communication protocols including, for example, any protocols conforming to any of the Ethernet standards, the Internet, or any combination thereof.

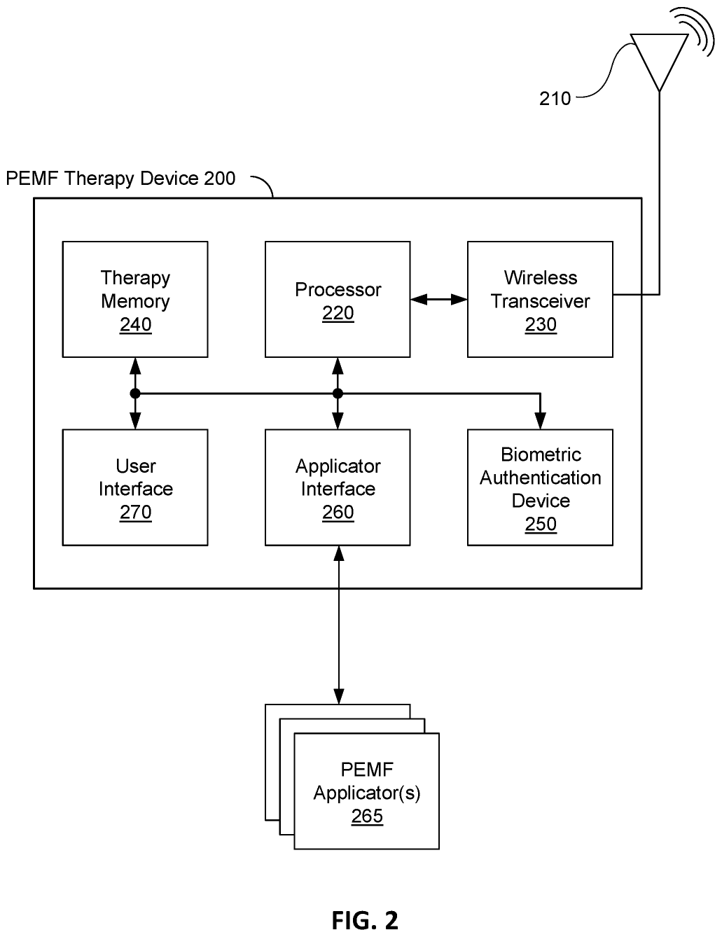

The biometric authentication device 114 may be any feasible device that can be used to verify a patient's identity. For example, the biometric authentication device 114 may compare one or more physical characteristics of a patient wishing to use the PEMF therapy device to the patient's previously stored physical characteristics. If the physical characteristics match, then the patient's identity if verified and the patient may be allowed to use the PEMF therapy device 110. On the other hand, if the physical characteristics do not match, then the patient may not be allowed to use the PEMF therapy device. The biometric authentication device is described in more detail below in conjunction with FIG. 2.

The wireless transceiver 122 of the treatment coordination server 120 may be coupled to the antenna 121. The wireless transceiver 122 may wirelessly communicate with other devices including, but not limited to, the PEMF therapy device 110. Wireless communication may be through one or more wireless protocols. For example, the wireless transceiver 122 may be configured to communicate via networks conforming to any of the IEEE 802.11 family of standards, cellular networks conforming to any of the LTE standards promulgated by the 3^{rd} Generation Partnership Project (3GPP) working group (e.g., 3G, 4G LTE, 5G LTE, or the like), WiMAX networks, Bluetooth networks, or the like. Additionally, or alternatively, the wireless transceiver 122 may be configured to communicate through any feasible wired communication protocols including, for example, any protocols conforming to any of the Ethernet standards, the Internet, or any combination thereof.

The treatment coordination server 120 may communicate with the PEMF therapy device 110. For example, the treatment coordination server 120 may communicate with the PEMF therapy device 110 using the wireless transceivers 122 and 112, contained therein. In some other examples, the treatment coordination server 120 may receive data regarding a patient's identity from the PEMF therapy device 110 (the patient's identity may be provided by the biometric authentication device 114). The treatment coordination server 120 may authorize treatment of the identified patient as well as provide patient treatment parameters (signal strength, applicator type, etc.) to the PEMF therapy device 110.

The patient treatment database 124 may store one or more treatment parameters of a patient's therapy (e.g., a patient's PEMF treatment plan) that may be delivered by the PEMF therapy device 110. For example, the patient treatment database 124 may include a patient's identity, a PEMF applicator type to be used to deliver a patient's PEMF therapy, an energy level associated with a high-power pulsed electromagnetic field signal that is associated with patient's PEMF therapy, a therapy duration (e.g., an amount of time that the high-power pulsed electromagnetic field signal is provided to the PEMF applicator), and the like.

The patient treatment database 124 may be determined, stored and/or updated by an authorized clinician. For example, a clinician caring for the patient may prescribe a particular PEMF treatment (applicator, duration, energy level, and the like). The clinician may store the PEMF treatment in the patient treatment database 124 through the wireless transceiver 122, a wired transceiver (not shown) or any other feasible interface. The clinician may also update and modify the patient's treatment in the patient treatment database 124 in a similar manner. For example, the patient's treatment may be modified according to changing physiological conditions of the patient.

To receive therapy from the PEMF therapy device 110, the patient's identity is first verified through the biometric authentication device 114. For example, the patient may present a biological identifier (e.g., finger, face, voice, eye, or the like) to the biometric authentication device 114. The biometric authentication device 114 may compare the presented biological identifier with stored biological identifier characteristics. If the presented biological identifier matches the stored characteristics, then the patient's identity is said to be verified.

The verified patient's identity may be encrypted and transmitted to the treatment coordination server 120. The treatment coordination server 120 can determine if a PEMF treatment corresponding to the verified patient is stored in the patient treatment database 124. If a corresponding treatment is found, then the treatment coordination server 120 may encrypt and transmit the treatment to the PEMF therapy device 110. The PEMF therapy device 110 may then deliver the PEMF therapy to the patient. In some cases, encryption of the patient's identity and the corresponding treatment may ensure the patient's privacy and conform to any Health Insurance Portability and Accountably Act (HIPPA) requirements. In some examples, the PEMF therapy device 110 and the treatment coordination server 120 may use public/private key encryption, Advanced Encryption Standard (AES), Rivest-Shamir-Alleman (RSA), Triple Data Encryption Standard (TripleDES), or any other feasible encryption to encrypt communications.

In this manner, the PEMF therapy device 110 may identify and authenticate a patient, retrieve a patient's PEMF therapy, and deliver this therapy to the patient. The biometric identification and verification may reduce the possibility of misuse or unauthorized use of the PERM therapy device. Furthermore, any updates to a patient's PEMF therapy treatment may be easily provided to the patient.

FIG. 2 depicts a PEMF therapy device 200 that may include an antenna 210, a processor 220, a wireless transceiver 230, a therapy memory 240, a biometric authentication device 250, an applicator interface 260, and a user interface 270. The PEMF therapy device 200 may be another example of the PEMF therapy device 110 of FIG. 1. Thus, the wireless transceiver 230 may be another example of the wireless transceiver 112, the antenna 210 may be another example of the antenna 111, and the biometric authentication device 250 may be another example of the biometric authentication device 114.

The processor 220 may be coupled to the wireless transceiver 230, the therapy memory 240, the biometric authentication device 250, the applicator interface 260, and the user interface 270. Thus, the processor 220 may control operations of the PEMF therapy device 200 by controlling and coordinating operations of the wireless transceiver 230, the therapy memory 240, the biometric authentication device 250, the applicator interface 260, and the user interface 270.

The processor 220 may communicate with other devices through the wireless transceiver 230 and the antenna 210. The wireless transceiver 230 may include any feasible transmitting/receiving radio. For example, the wireless transceiver 230 may include one or more radios configured to wirelessly communicate using any feasible protocol including, but not limited to, cellular (e.g., 3G, 4G LTE, 5G LTE, or the like), Wi-Fi (e.g., any of the IEEE 802.11 family of standards), Bluetooth, or other protocols.

The wireless transceiver 230 may communicate with a treatment coordination server (not shown). In some examples, the PEMF therapy device 200 may receive an authorization from the treatment coordination server to deliver a PEMF therapy to a patient. Additionally, the treatment coordination server may provide a patient's treatment plan information including, for example, PEMF applicator type, therapy duration, patient identity, energy level associated with the high-power pulsed electromagnet field signal associated with the PEMF therapy, and the like.

Additionally, or alternatively, the wireless transceiver 230 may communicate with one or more patient devices using Bluetooth protocols. In one example, the patient may wear a fitness tracking device. The PEMF therapy device 200 may receive patient information (pulse rate, temperature, sleep cycle information, and the like) from the patient's fitness tracking device before, during, or after a PEMF treatment is delivered. The PEMF therapy device 200 may, in turn, transmit the patient's information to the treatment coordination server.

The therapy memory 240 may store a patient's PEMF treatment plan received from the treatment coordination server. As described above, a patient's PEMF treatment plan may include, but is not limited to, information regarding PEMF applicator type, therapy duration, patient identity, and energy level associated with the high-power pulsed electromagnet field signal associated with the PEMF therapy. In some examples, the patient's information (for example, from a fitness tracking device) may also be stored in the therapy memory 240.

The biometric authentication device 250 may be any feasible device to verify the identity of a patient or user of the PEMF therapy device 200. In some examples, the biometric authentication device 250 may include a fingerprint scanner, a camera, a voice/speech recognition device, a retina scanner, or the like. The biometric authentication device 250 may store, in a secure memory, characteristics of the biologic features that are used to verify or identify the patient.

In one example, the biometric authentication device 250 may include a fingerprint scanner (not shown). Prior to receiving a first PEMF therapy, the patient can “enroll” one or more finger or thumbprints with the fingerprint scanner. Characteristics of the enrolled finger or thumbprints may be stored in a secure memory (not shown) accessible to the biometric authentication device 250. After enrollment, when the patient wishes to use the PEMF therapy device 200, the patient presents his/her finger or thumb to the fingerprint scanner. The fingerprint scanner can compare characteristics (e.g., ridges, valleys, and the like) of the scanned finger or thumbprint to the stored fingerprint characteristics to determine or verify the identity of the patient.

In another example, the biometric authentication device 250 may include a camera (not shown). Prior to receiving a first PEMF therapy, the patient can enroll with the biometric authentication device 250 by presenting one or more facial images to the camera. Characteristics of the enrolled facial image may be stored in a secure memory (not shown) accessible to the biometric authentication device 250. After enrollment, when the patient wishes to use the PEMF therapy device 200, the patient presents his/her face to the camera for facial recognition. The biometric authentication device 250 can compare characteristics of the face presented to the camera to stored enrollment characteristics to determine or verify the identity of the patient.

In another example, biometric authentication device 250 may include a retinal scanner (not shown). Prior to receiving a first PEMF therapy, the patient can enroll with the biometric authentication device 250 by presenting one or more retinal images to the retinal scanner. Characteristics of the retinal image may be stored in a secure memory (not shown) accessible to the biometric authentication device 250. After enrollment, when the patient wishes to use the PEMF therapy device 200, the patient presents his/her retina to the retina scanner for a retinal scan. The biometric authentication device 250 can compare characteristics of the retina at the retina scanner to stored retinal characteristics to determine or verify the identity of the patient.

In another example, biometric authentication device 250 may include a palm scanner (not shown). Prior to receiving a first PEMF therapy, the patient can enroll with the biometric authentication device 250 by presenting one or more palm images to the palm scanner. Characteristics of the palm images may be stored in a secure memory (not shown) accessible to the biometric authentication device 250. After enrollment, when the patient wishes to use the PEMF therapy device 200, the patient presents his/her palm to the palm scanner. The biometric authentication device 250 can compare characteristics of the palm at the palm scanner to stored palm characteristics to determine or verify the identity of the patient.

In yet another example, biometric authentication device 250 may include a voice recognition device (not shown). Prior to receiving a first PEMF therapy, the patient can enroll with the biometric authentication device 250 by presenting one or more identification phrases to the voice recognition device. Characteristics of the identification phrase may be stored in a secure memory (not shown) accessible to the biometric authentication device 250. After enrollment, when the patient wishes to use the PEMF therapy device 200, the patient speaks the identification phrase to the voice recognition device. The biometric authentication device 250 can compare characteristics of the voice at the voice recognition device to stored voice characteristics to determine or verify the identity of the patient.

The examples of the biometric authentication device 250 described herein are merely to illustrate example devices and are not meant to be limiting. The biometric authentication device 250 may be implemented with any other feasible device. Notably, the patient biometric characteristics are stored locally in a secure memory. That is, a patient's physical characteristics remain stored within the secure memory of the PEMF therapy device 200 and are never transmitted to any other device. Thus, sensitive patient data is maintained secure, easing conformance with any possible HIPPA requirements.

The processor 220 may control the delivery of electromagnetic field therapies through the applicator interface 260. One or more PEMF applicators 265 may be coupled to the applicator interface 260. In some examples, the applicator interface 260 may include drive circuitry to provide high-power pulsed electromagnetic field voltage and/or current signals that enable the one or more PEMF applicators 265 to emit electromagnetic fields (e.g., electric and/or magnetic fields). These electromagnetic fields may be used to deliver therapeutic treatment to the patient.

In some examples, the applicator interface 260 may also include circuitry to communicate with and/or identify the coupled PEMF applicators 265. For example, the applicator interface 260 may include serial communication circuitry to communicate with the PEMF applicators 265. Through this circuitry, the PEMF therapy device 200 may determine, for example, make, model, and manufacturer of the coupled PEMF applicators 265. In one example, operation of the PEMF therapy device 200 may be inhibited if the applicator interface 260 fails to identify an appropriate PEMF applicator 265, for example, as specified in the patient's PEMF treatment plan stored in the therapy memory 240.

The user interface 270 may include one or more input or output devices that enable the patient to interact with the PEMF therapy device 200. For example, the user interface 270 may include a display that enables the display of device status or provides images and/or instructions to the patient guiding use of the PEMF therapy device 200. In another example, the user interface 270 may include a keyboard, mouse, touch screen, touch pad, buttons, or the like, to receive input from the patient. In still another example, the user interface 270 may include a speaker for the reproduction of voice instructions for the patient.

In some examples, the user interface 270 may be used to enroll the patient in conjunction with the biometric authentication device 250. For example, the PEMF therapy device 200 may communicate with the treatment coordination server to begin enrollment of a patient. In response, the treatment coordination server may transmit a Quick Response (QR) code for display on the user interface 270. The patient may scan the QR code with a smartphone. The patient's smartphone may communicate with the PEMF therapy device 200 (via Bluetooth or Wi-Fi, for example) or with the treatment coordination server (through LTE, for example) to confirm the QR code displayed on the PEMF therapy device 200.

In some examples, the patient may use the user interface 270 to enter a personal identification (PIN) code to access and use the PEMF therapy device 200. For example, the user interface 270 may display a prompt asking the patient to verify his/her identity. Instead of (or in parallel with) using the biometric authentication device 250, the user may verify his/her identity by supplying a PIN code. In some examples, the user may enter the PIN code via a keypad, touch screen, or any other feasible device.

### Example Use of PEMF Therapy Device

Prior to receiving PEMF therapy, a clinician may determine and store a patient's PEMF therapy information in the treatment coordination server. Furthermore, prior to receiving PEMF therapy, the patient may have already enrolled with the biometric authentication device 250. Thus, to begin treatment, the patient may verify his/her identity with the biometric authentication device 250. If the patient's identity is verified, then the patient's identity may be transmitted to the treatment coordination server. In some examples, the patient identity may be encrypted prior to transmission.

After the treatment coordination server receives the patient identity, the treatment coordination server may retrieve the stored patient's PEMF treatment plan (for example, stored in the patient treatment database 124 of FIG. 1). In some examples, the patient's PEMF treatment plan may include information including the patient's identity, the PEMF applicator type to be used to deliver a patient's PEMF therapy, an energy level associated with a high-power pulsed electromagnetic field signal that is associated with patient's PEMF therapy, and a therapy duration. The treatment coordination server may transmit the patient's PEMF treatment plan to the PEMF therapy device 200. In some examples, the patient's PEMF treatment plan may be encrypted prior to transmission.

In some examples, the wireless connectivity is for the therapy and server system may be collocated within a building, a room, or a relatively close distance. In general, the server system could be located in a remote location. Examples of wireless features may include any wireless circuitry and/or antenna that may connect via Wi-Fi, Bluetooth, LTE/5G, satellite, or an alternative form of communication platform to connect with the remote server system via the Internet.

The PEMF therapy device 200 may receive the patient's PEMF treatment plan and determine if the appropriate PEMF applicator 265 is coupled to the applicator interface 260. After determining that the coupled PEMF applicator 265 is appropriate, the PEMF therapy device 200 can deliver the PEMF therapy as described by the patient's PEMF treatment plan.

Further, the treatment coordination server may have transmitted (and the PEMF therapy device 200 may have received) instructions to be displayed on a display included within the user interface 270. The instructions may include a description of the suggested PEMF applicators 265 to use as well as proper body placement of the PEMF applicator 265.

After receiving the treatment, the patient may provide self-assessment data to the PEMF therapy device 200 through the user interface 270. Self-assessment data may include a pain self-assessment. In addition, the PEMF therapy device 200 may receive a patient information from, for example, a patient's fitness tracking device or similar device. The self-assessment data and the patient information may be transmitted to the treatment coordination server. In some examples, the self-assessment data and the patient information may be encrypted prior to transmission.

In any of these apparatuses and methods, one or more (e.g., two, two or more, three, three or more, etc.) applicators may be used. For example, a PEMF device will have at least two pads to treat each foot or a pad capable of treating two feet.

A clinician can review the health assessment data and the patient information received by the treatment coordination server. In some cases, the clinician can update the patient's PEMF treatment plan and store the plan in the patient treatment database. In this manner, the patient's PEMF treatment plan may be updated before the next PEMF treatment session by the patient.

Use of the biometric authentication device 250 may restricted the use of the PEMF therapy device 200 to preapproved users/patients. In some examples, use of the PEMF therapy device 200 may be disabled remotely by a clinician. For example, if a patient reports that the PEMF therapy device 200 has been stolen or is missing, then the treatment coordination server may transmit instructions to the PEMF therapy device 200 to shut down or otherwise be incapable of providing any therapy.

### Example Methods for Providing PEMF Therapy

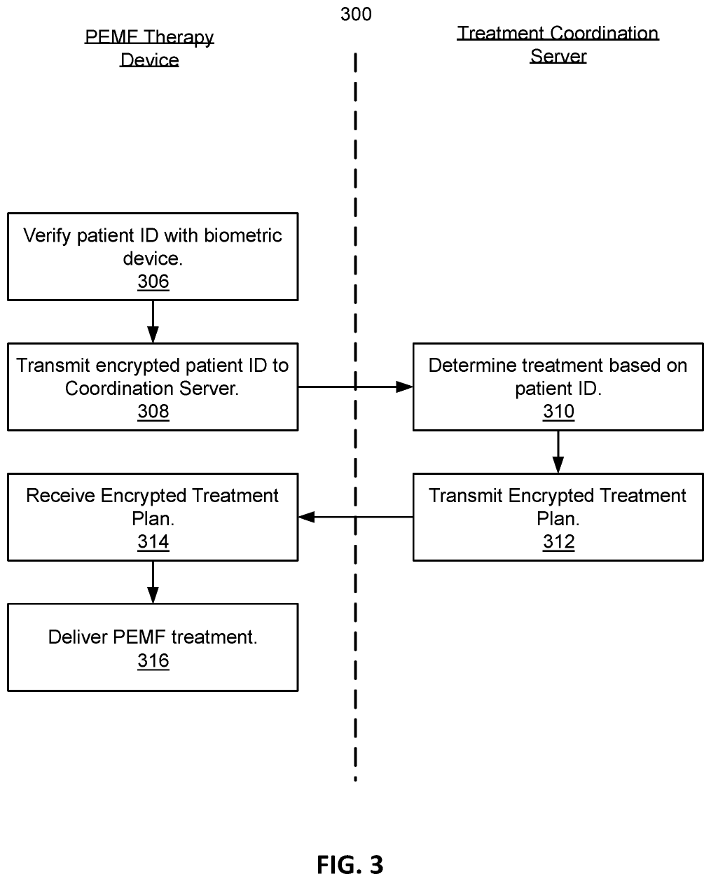

FIG. 3 is a flowchart depicting one example method 300 for providing a pulsed electromagnetic field therapy to a patient. Some other examples may perform the operations described herein with additional operations, fewer operations, operations in a different order, operations in parallel, and some operations differently. The operations herein are described as being performed by a PEMF therapy device and/or a treatment coordination server (for example, the PEMF therapy device 110 and treatment coordination server 120 of FIG. 1) for ease of explanation. Persons having skill in the art will recognize that the operations can be performed by any feasible device or processor that may be configured to receive and/or detect the conditions described herein and perform and/or deliver the therapies described herein.

In FIG. 3, the method 300 may begin as the PEMF therapy device verifies a patient's identity with a biometric subsystem 306. The patient's identity may be verified to help ensure that the PEMF therapy device is being used by an authorized patient. Furthermore, the patient's identity may be used to ensure that the correct therapy is delivered to the patient. In some examples, a patient may use a fingerprint scanner to verify his/her identity. In another example, the patient may use a facial recognition device to verify his/her identity. In other examples, the biometric subsystem may be a retinal scanner, a hand/palm scanner, a voice recognition device, or any other feasible biometric authentication device to verify the patient's identity. If the patient's identity is not verified, then the PEMF therapy device may not provide PEMF treatment.

Next, the PEMF therapy device may transmit an encrypted patient identity to the treatment coordination server 308. For example, once the biometric subsystem 306 has verified the identity of a patient, the PEMF therapy device can encrypt and transmit the verified patient identity to the therapy coordination server. Notably, the biologic characteristics of the patient that may be used to verify his/her identity are never transmitted and remain safely within a secure memory of the PEMF therapy device. In some examples, a patient identifier, which may anonymously and uniquely identify a patient, may be encrypted, and transmitted to the therapy coordination server.

Next, the therapy coordination server determines (e.g., recalls, identifies) the patient's PEMF treatment plan based on the received patient identity 310. For example, the therapy coordination server can receive the patient identity from the PEMF therapy device and, in response, can retrieve the appropriate patient PEMF treatment plan from the patient treatment database associated with the patient identity. The patient PEMF treatment plan may include a patient's identity, a PEMF applicator type, an energy level associated with a high-power pulsed electromagnetic field signal that is associated with patient's PEMF therapy, and a therapy duration.

Next, the therapy coordination server may transmit the patient PEMF treatment plan to the PEMF therapy device 312. In some examples, the patient PEMF treatment plan retrieved from the patient treatment database may be encrypted and transmitted to the PEMF therapy device.

Next, the PEMF therapy device can receive the patient's PEMF treatment plan 314 and deliver the PEMF treatment 316. In some examples, the PEMF therapy may be delivered through the PEMF applicators coupled to the PEMF therapy device.

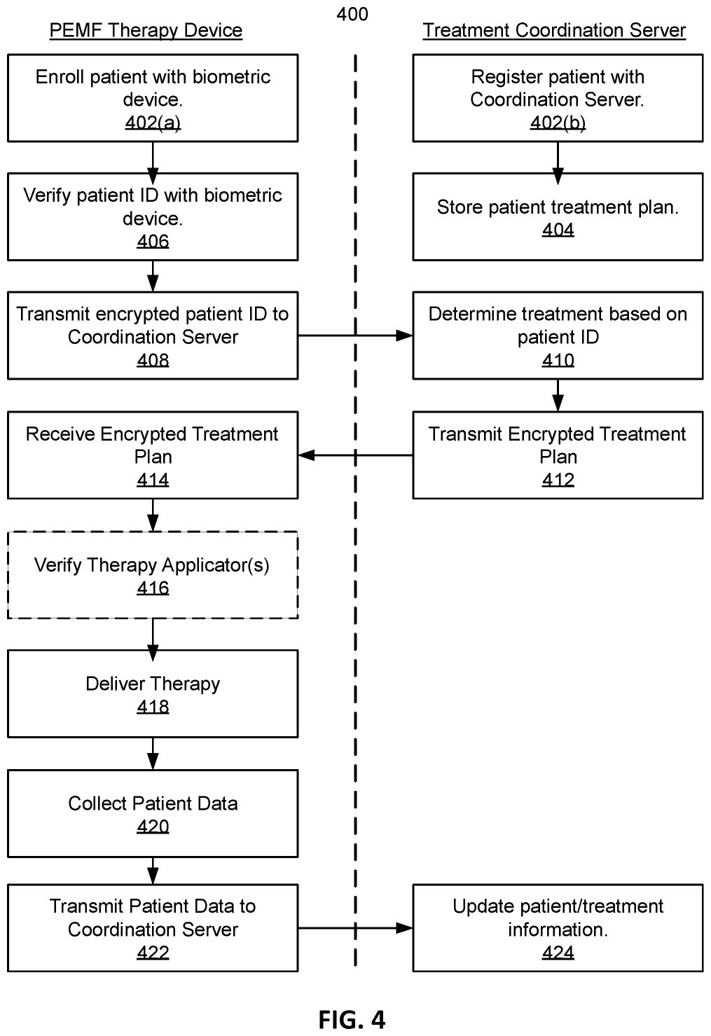

FIG. 4 is a flowchart depicting another example method 400 for providing a pulsed electromagnetic field therapy to a patient. Some other examples may perform the operations described herein with additional operations, fewer operations, operations in a different order, operations in parallel, and some operations differently. The method of FIG. 4 may be similar to the method of FIG. 3 and may include additional operations to deliver PEMF therapy.

The method 400 may begin as a patient enrolls with a biometric subsystem at the PEMF therapy device 402a. Additionally, the patient may be registered with the treatment coordination server 402(b). Although shown as being performed at approximately the same time, persons having skill in the art will recognize that these steps may be performed independently and not necessarily concurrently or within any time restrictions.

To enroll with some biometric subsystems, the patient may present multiple biologic features that may be scanned or captured. For example, if the biometric subsystem is a fingerprint scanner, the patient may present one or more fingers multiple times to the fingerprint scanner so that the fingerprint scanner can “learn” the biometric characteristics associated with the patient's fingerprints. If the biometric subsystem is a facial recognition device, then the facial recognition device may capture one or more images of the patient's face so that the device can learn the biometric characteristics associated with the patient's face. If the biometric subsystem is a hand or palm scanner, then the patient may present his/her palm one or more times so that the hand/palm scanner can learn the biometric characteristics associated with the patient's hand. If the biometric subsystem is a retinal scanner, then the patient may present one or more eyes to the retinal scanner so that the retinal scanner can learn the biometric characteristics associated with the patient's eye. If the biometric subsystem is a voice recognition device, then the patient may speak one or more phrases to train the voice recognition device for the patient's voice. The biometric subsystems described here are exemplary and not meant to be limiting. In some cases, the patient's enrollment with the PEMF therapy device may be performed with two factor authentication that may involve a QR codes, pin codes, and the like being transmitted between the PEMF therapy device and the patient coordination server.

As noted above, the patient may also be registered with the treatment coordination server 402(b). Thus, patient records and/or files may be established in the treatment coordination server that correspond to the patient enrolled at the PEMF therapy device.

Next, the patient's PEMF treatment plan may be stored in the treatment coordination server 404. For example, a clinician having assessed the patient, may prescribe a particular PEMF treatment plan. The patient's PEMF treatment plan may include a patient's identity, a PEMF applicator type to be used to deliver a patient's PEMF therapy, an energy level associated with a high-power pulsed electromagnetic field signal that is associated with patient's PEMF therapy, a therapy duration (e.g., an amount of time that the high-power pulsed electromagnetic field signal is provided to the PEMF applicator), and the like.

Next, for the patient to receive PEMF therapy, the patient verifies his/her identity with the biometric subsystem 406. For example, the patient may present fingerprints, eye scans, face, etc., to the biometric subsystem on the PEMF therapy device to verify the patient's identity. It the patient's identity is not verified, then no PEMF therapy may be delivered. Next, after the patient's identity is verified, then the PEMF therapy device may transmit the verified patient's identity to the treatment coordination server 408. In some examples, the PEMF therapy device may encrypt the patient's identity before being transmitted. Next, the treatment coordination server can determine the PEMF treatment based on the patient's identity 410. For example, the treatment coordination server can receive the encrypted patient's identity, decrypt the patient's identity, and then retrieve or recall the patient's PEMF treatment plan for the patient from a therapy treatment database. As described above, the patient's PEMF treatment plan may include a specification of a patient's identity, a PEMF applicator, a PEMF therapy duration, an energy level associated with a high-power pulsed electromagnetic field signal that is associated with patient's PEMF therapy, and/or other aspects of a PEMF therapy.

Next, the therapy treatment coordination server may transmit the patient's PEMF treatment plan to the PEMF therapy device 412. In some examples, the treatment coordination server may encrypt the patient's PEMF treatment plan before transmission. Next, the PEMF therapy device may receive the patient's PEMF treatment plan from the treatment coordination server 414. In some examples, the PEMF therapy device may decrypt the patient's PEMF treatment plan.

Next, the PEMF therapy device can verify the PEMF therapy applicator coupled to the PEMF therapy device 416. This step may be optional as shown by the dashed lines in FIG. 4. In some cases, the patient's PEMF treatment plan received from the therapy coordination server may specify a particular PEMF applicator to be used. Therefore, the PEMF therapy device can determine whether the appropriate PEMF applicator is coupled to the PEMF therapy device. In some examples, the PEMF therapy device may also determine if an approved PEMF applicator is coupled to the PEMF therapy device. For example, a third party PEMF applicator may be coupled to the PEMF therapy device. While the third party PEMF applicator may be similar to the approved or official PEMF applicator, the third party PEMF applicator may not be approved for use by the clinician and/or the PEMF therapy device manufacturer. Therefore, the PEMF therapy device may determine that the correct and appropriate PEMF applicator is coupled prior to beginning PEMF therapy. If the correct, approved PEMF applicator is not detected, then the PEMF therapy may not be provided by the PEMF therapy device.

Next, the PEMF therapy device can deliver PEMF therapy to the patient 418. For example, the PEMF therapy device may deliver the PEMF therapy as described in the patient's PEMF treatment plan received from the therapy coordination server at 414. Next, the PEMF therapy device may collect patient data 420. For example, the PEMF therapy device may receive a patient self-assessment regarding patient pain levels, or any other patient reported symptoms. In some examples, the patient may provide the self-assessment information or pain assessment information through an included user interface or through another device such as a smartphone that may be running an application to collect patient information. In some examples, the PEMF therapy device may also collect patient data from one or more patient peripherals. For example, the PEMF therapy device may collect patient data from a patient's fitness tracking device as the PEMF therapy is being delivered in 418.

Next, the patient data may be transmitted to the coordination therapy server 422. For example, the patient self-assessment, patient data from fitness tracking devices, and/or pain data provided by the patient may be encrypted and transmitted to the therapy coordination server.

Next, the patient's PEMF treatment plan may be updated at the therapy coordination server 424. For example, a clinician can review the patient's self-assessment data, pain data, and the patient data collected from the patient's fitness tracking devices. Upon review, the clinician may determine that the patient's PEMF treatment plan should updated to reflect changes in the patient's condition. The updated therapy can be stored in the patient treatment database. In this manner, the next time that the patient uses the PEMF therapy device, the updated PEMF treatment plan may be provided to the PEMF therapy device at 412.

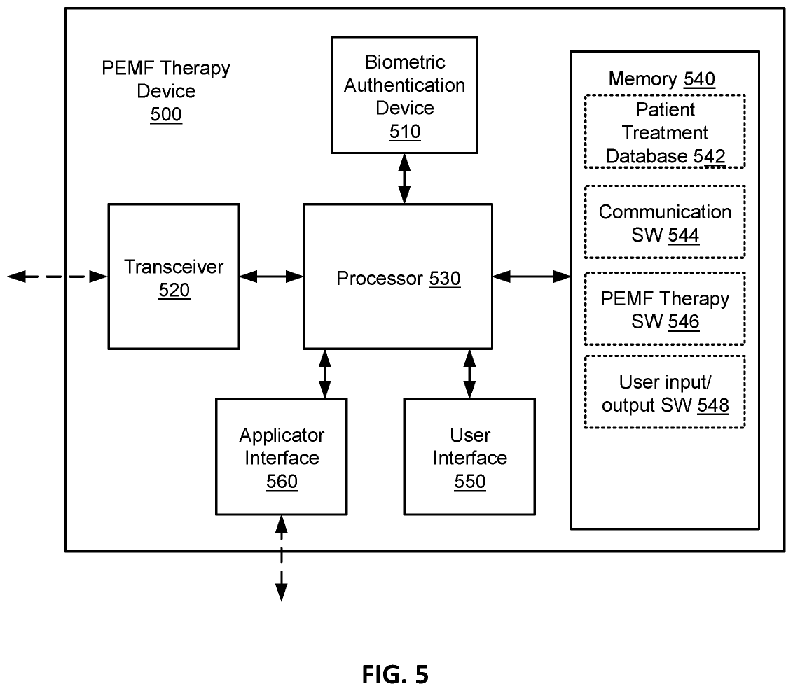

FIG. 5 shows a block diagram of a PEMF therapy device 500 that may be one example of the PEMF therapy device 110 of FIG. 1 or PEMF therapy device 200 of FIG. 2. The PEMF therapy device 500 may include a biometric authentication device 510, a transceiver 520, an applicator interface 560, a memory 540, and a user interface 550.

The biometric authentication device 510, which is coupled to the processor 530, may be used to verify the identity of a patient that wishes to use the PEMF therapy device 500. The biometric authentication device 510 may be another example of the biometric authentication device 114 of FIG. 1 and/or the biometric authentication device 250 of FIG. 2. The biometric authentication device 510 may include a fingerprint scanner, a camera, a voice/speech recognition device, a retina scanner, or any other feasible biometric identification device. The biometric authentication device 510 may include a secure memory (not shown) for storing biometric characteristics that may be used to identify different patients.

The transceiver 520 may be used to transmit signals to and receive signals from other devices. The transceiver 520 may be coupled to an antenna (not shown) and may wirelessly communicate with any other feasible device. The applicator interface 560 may couple the PEMF therapy device 500 to one or more PEMF applicators (not shown). In some examples, the applicator interface (which may be an example of the applicator interface 260) may include communication circuitry to communicate with the one or more PEMF applicators and may also include driver circuitry to generate high-power pulsed electromagnet field signals for the PEMF applicators. The user interface 550 (which may be an example of the user interface 270), may include a keyboard, mouse, touch screen, touch pad, buttons, or the like to enable a patient to interact with and provide data to the PEMF therapy device 500.

The memory 540 may include a patient treatment database 542 that may be used to locally store PEMF treatment plans for one or more patients. For example, the patient treatment database 542 may include a PEMF applicator type to be used to deliver a patient's PEMF therapy, an energy level associated with a high-power pulsed electromagnetic field signal that is associated with patient's PEMF therapy, a therapy duration (e.g., an amount of time that the high-power pulsed electromagnetic field signal is provided to the PEMF applicator), and the like.

The memory 540 may also include a non-transitory computer-readable storage medium (e.g., one or more nonvolatile memory elements, such as EPROM, EEPROM, Flash memory, a hard drive, etc.) that may store the following software modules:
a communication software (SW) module 544 to communicate with other devices through the transceiver 520;
a PEMF therapy SW module 546 to deliver PEMF therapy through PEMF applicators coupled to the applicator interface 560; and
a user input/output SW module 548 to receive and provide user input and output through the user interface 550.
   Each software module includes program instructions that, when executed by the processor 530, may cause the PEMF therapy device 500 to perform the corresponding function(s). Thus, the non-transitory computer-readable storage medium of memory 540 may include instructions for performing all or a portion of the operations described herein.

The processor 530, which is coupled to the biometric authentication device 510, the transceiver 520, the applicator interface 560, the memory 540, and the user interface 550, may be any one or more suitable processors capable of executing scripts or instructions of one or more software programs stored in the PEMF therapy device 500 (e.g., within the memory 540).

The processor 530 may execute the communication SW module 544 to communicate with any other feasible devices. For example, execution of the communication SW module 544 may enable the PEMF therapy device 500 to communicate via cellular networks conforming to any of the LTE standards promulgated by the 3^{rd} Generation Partnership Project (3GPP) working group, Wi-Fi networks conforming to any of the IEEE 802.11 standards, Bluetooth protocols put forth by the Bluetooth Special Interest Group (SIG), Ethernet protocols, or the like. In some examples, execution of the communication SW module 544 may enable the PEMF therapy device 500 to communicate with a therapy coordination server and/or a fitness tracking device (not shown). In some other examples, execution of the communication SW module 544 may implement encryption and/or decryption procedures. Further, the PEMF therapy device 500 may transmit verified patient identity information from the biometric authentication device 510 to the therapy coordination server.

In some examples, execution of the communication SW module 544 may enable the PEMF therapy device 500 to receive a patient's PEMF treatment plan and store the plan in the patient treatment database 542. Further, execution of the communication SW module 544 may enable the PEMF therapy device 500 to transmit patient info (e.g., self-assessment information, fitness tracking device information, and the like) to the therapy coordination server.

The processor 530 may execute the user input/output SW module 548 to receive user input such as self-assessment information, pain assessment information, and PIN code information. Additionally, execution of the user input/output SW module 548 may enable the PEMF therapy device 500 to provide the patient information including, for example, details regarding the patient's PEMF treatment plan, directions for using the PEMF therapy device, or any other feasible information.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of controlling operation of a pulsed electromagnetic field (PEMF) therapy device, the method comprising:
verifying a patient's identity via a biometric subsystem of the PEMF device;
transmitting the verified patient's identity to a treatment coordination server;
receiving a PEMF treatment plan associated with the transmitted verified patient's identity from the treatment coordination server; and
delivering the PEMF treatment to the patient according to the received PEMF treatment plan.

2. The method of claim 1, wherein the biometric subsystem includes one or more of: a fingerprint scanner, a retina scanner, a facial recognition device, a voice recognition device, a hand scanner, or a combination thereof.

3. The method of claim 1, wherein transmitting comprises wireless transmitting.

4. The method of claim 1, wherein the transmitting the verified patient's identity includes encrypting the verified patient's identity.

5. The method of claim 1, wherein the received PEMF treatment plan includes a PEMF applicator type, a therapy duration, and an energy level associated with the PEMF therapy.

6. The method of claim 1, wherein receiving the PEMF treatment plan further comprises decrypting the PEMF treatment plan from the treatment coordination server.

7. The method of claim 1, further comprising verifying one or more PEMF therapy applicators coupled to the PEMF device prior to delivering the PEMF treatment.

8. The method of claim 7, wherein the PEMF treatment is not delivered when the one or more PEMF therapy applicators coupled to the PEMF therapy device are not included in the PEMF treatment plan.

9. The method of claim 1, further comprising collecting patient data via the PEMF therapy device.

10. The method of claim 9, wherein the patient data includes self-assessment data, pain data, patient pulse rate, patient temperature, patient sleep cycle information, or a combination thereof.

11. The method of claim 9 further comprising transmitting the patient data to the treatment coordination server.

12. The method of claim 11, further comprising updating the PEMF treatment plan based on received patient data.

13. The method of claim 1, further comprising disabling the PEMF device based on verified patient's identity.

14. A method of controlling operation of a pulsed electromagnetic field (PEMF) therapy device, the method comprising:
receiving an indicator of a patient's identity via a biometric subsystem of the PEMF device;
receiving an encrypted accessory authentication indicator for one or more applicators coupled to the PEMF device;
transmitting the indicator of the patient's identity, an indicator of an identity of the PEMF therapy device, and the encrypted accessory authentication indicator to a treatment coordination server;
receiving a PEMF treatment plan associated with the patient's identity from the treatment coordination server upon verification by the treatment coordination server that the patient identity is linked to the one or more applicators and to the PEMF device; and
delivering the PEMF treatment to the patient.

15. The method of claim 14, further comprising verifying the patient's identity in the PEMF device using the indicator of the patient's identity before transmitting the indicator of the patient's identity to the treatment coordination server.

16. The method of claim 14, further comprising verifying the patient's identity in the treatment coordination server using the indicator of the patient's identity.

17. The method of claim 14, further wherein the biometric subsystem includes one or more of: a fingerprint scanner, a retina scanner, a facial recognition device, a voice recognition device, a hand scanner, or a combination thereof.

18. The method of claim 14, wherein the transmitting the indicator of the patient's identity includes encrypting the indicator of the patient's identity.

19. The method of claim 14, wherein the received PEMF treatment plan includes a therapy duration, and an energy level associated with the PEMF therapy.

20. The method of claim 14, wherein receiving the PEMF treatment plan further comprises decrypting the PEMF treatment plan.

21. The method of claim 14, further comprising receiving a notification that the patient's identity is not linked to the one or more applicators and/or to the PEMF device.
